(19) United States
(12) Patent Application Publication
(10) Pub. No.: US 2013/0060241 A1
(43) Pub. Date: Mar. 7, 2013

(54) DYNAMIC REAL TIME ACTIVE PUPIL CENTROID COMPENSATION

(71) Applicant: Daniel S. Haddad, Bloomfield Hills, MI (US)

(72) Inventor: Daniel S. Haddad, Bloomfield Hills, MI (US)

(21) Appl. No.: 13/641,934

(22) PCT Filed: Apr. 22, 2011
(86) PCT No.: PCT/US11/33595

§ 371 Date: Nov. 19, 2012

Related U.S. Application Data
(60) Provisional application No. 61/328,414, filed on Apr. 27, 2010.

(51) Int. Cl. A61B 18/20; A61B 3/11

(52) U.S. Cl. 606/4; 351/205; 351/206; 351/246

(57) ABSTRACT

An apparatus and method for actively compensating for pupil centroid shift includes obtaining a first measurement of a first reference point relative to a predetermined reference frame, wherein the first reference point being associated with a first pupil diameter of a patient. The method further includes obtaining a second measurement of a second reference point relative to the predetermined reference frame, wherein the second reference point is associated with a second pupil diameter of the patient and the second pupil diameter is different from the first pupil diameter. The method still further includes actively determining a relationship between the first measurement and the second measurement and actively generating a correction in response to the relationship, wherein the correction being used by a treatment laser in association with an eye surgery.

### CROSS-REFERENCE TO RELATED APPLICATIONS

This application claims the benefit of U.S. Provisional Application No. 61/328,414, filed on Apr. 27, 2010. The entire disclosure of the above application is incorporated herein by reference.

### FIELD

The present disclosure relates to vision correction systems and methods and more particularly to pupil centroid shift compensation systems and methods.

### BACKGROUND AND SUMMARY

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

Vision correction treatments generally treat the cornea of an eye to correct one or more refractive errors of the eye. For example only, a laser may be used to treat the cornea in laser-assisted in situ keratomileusis (LASIK), laser-assisted sub-epethilial keratectomy (LASEK), and photorefractive keratectomy (PRK) vision correction treatments.

Prior to the treatment, an optical measuring device, such as a refractometer (e.g., an auto-refractor, a pupilometer, etc.) or an aberrometer (e.g., a wavefront aberrometer), may be used to measure refractive errors and aberrations of the optical system. The refractometer or aberrometer may also determine one or more parameters, such as pupil centroid. A treatment plan for the procedure may be generated based on data from such optical measuring devices, like the aberrometer, and other data.

Pupil centroid may refer to a center location of the pupil of the eye with respect to a reference location. The reference location may include, for example, a center location of the pupil when fully dilated, a center of an outside of the cornea of the eye, or another suitable reference location. The pupil centroid may be expressed two-dimensionally (e.g., X and Y) with respect to the reference location. The pupil centroid may be expressed three-dimensionally in various implementations (e.g., X, Y, and Z) with respect to the reference location.

### BRIEF DESCRIPTION OF THE DRAWINGS

The present disclosure will become more fully understood from the detailed description and the accompanying drawings, wherein:

FIG. 1 is an exemplary illustration of an eye according to the principles of the present disclosure;

FIG. 2 is an exemplary illustration of pupil centroid shift according to the principles of the present disclosure;

FIG. 3 is an exemplary illustration of a vision correction procedure that is centered versus a vision correction procedure that is decentered according to the principles of the present disclosure;

FIG. 4 is a functional block diagram of an exemplary pupil centroid shift determination and storage system according to the principles of the present disclosure;

FIG. 5 is an exemplary graph of pupil centroid shift as a function of pupil size and an exemplary illustration of pupil size and pupil centroid shift as functions of light intensity according to the principles of the present disclosure;

FIG. 6 is a functional block diagram of an exemplary vision treatment system according to the principles of the present disclosure; and

FIG. 7 is a flowchart depicting an exemplary method of accounting for pupil centroid shift in real time during a vision correction treatment according to the principles of the present disclosure.

### DETAILED DESCRIPTION

The following description is merely exemplary in nature and is in no way intended to limit the disclosure, its application, or uses. For purposes of clarity, the same reference numbers will be used in the drawings to identify similar elements. As used herein, the phrase at least one of A, B, and C should be construed to mean a logical (A or B or C), using a non-exclusive logical or. It should be understood that steps within a method may be executed in different order without altering the principles of the present disclosure.

As used herein, the term module refers to an Application Specific Integrated Circuit (ASIC), an electronic circuit, a processor (shared, dedicated, or group) and memory that execute one or more software or firmware programs, a combinational logic circuit, and/or other suitable components that provide the described functionality.

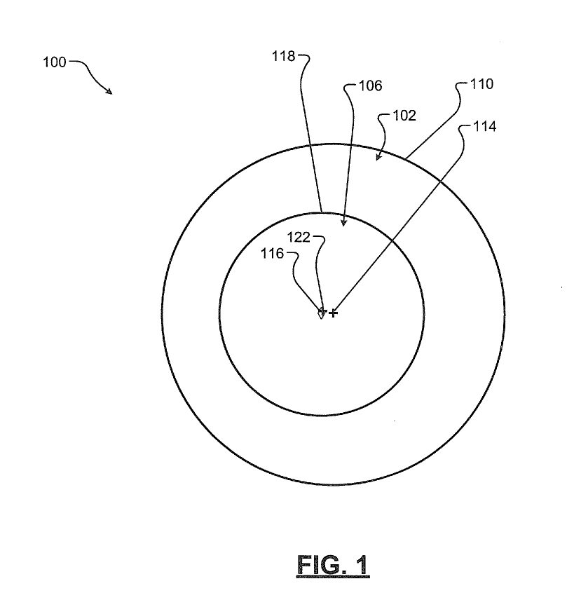

Referring now to FIG. 1, an exemplary image 100 of a human eye is presented. Among other things, the eye includes an iris 102 and a pupil 106. The pupil 106 is an aperture in the iris 102. While not numbered, a cornea covers the iris 102 and the pupil 106. A vision correction treatment, such as a laser-assisted in situ keratomileusis (LASIK) treatment, a laser-assisted sub-epethilial keratectomy (LASEK) treatment, a photorefractive keratectomy (PRK) treatment, or another suitable type of vision correction treatment may treat the cornea or treat a lens within the pupil.

More specifically, a vision correction treatment may involve treating one or more portions of the cornea. For purposes of discussion only, exemplary circular trace 110 may be said to correspond to an outside of the iris 102. However, the exemplary circular trace 110 may correspond to a limbus of the eye (i.e., a border where the cornea and a sclera meet). Exemplary plus-shaped mark 114 may correspond to a center location of the circular trace 110 (i.e., the center of the iris 102). Exemplary diamond-shaped mark 116 may correspond to a line of sight. For purposes of discussion only, exemplary circular trace 118 may be said to correspond to an outer perimeter of the pupil 106, but the circular trace 118 may also correspond to an inner perimeter of the iris 102. Exemplary plus-shaped mark 122 may correspond to a center location of the circular trace 114 (i.e., the center of the pupil 106).

Pupil centroid may refer to a center location of the pupil 118 with respect to a first reference location. For example only, the first reference location may be the center of the iris 110 or another suitable reference location.

The pupil centroid may be expressed as a multi-dimensional coordinate that relates the center of the pupil 118 to the first reference location. For example only, the pupil centroid may be expressed as a two-dimensional coordinate (e.g., X and Y) or a three-dimensional coordinate (e.g., X, Y, and Z) that relates the center of the pupil to the first reference location. The pupil centroid may be expressed in three-dimensions, for example, to account for parallax.

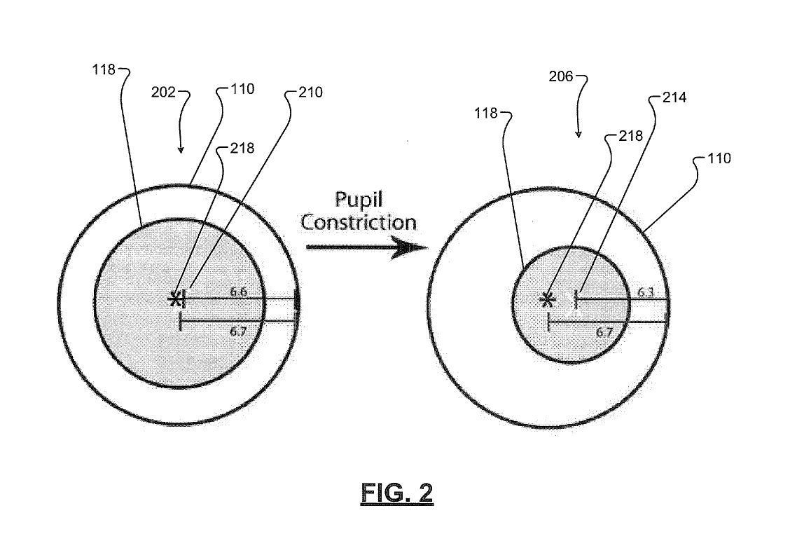

Referring now to FIG. 2, an exemplary illustration of pupil centroid shift is presented. During performance of a vision correction treatment, a laser or another treatment device may be centered based on the pupil centroid. However, it has been found that the pupil centroid may vary with pupil dilation, constriction, and/or other factors. For example only, the pupil centroid may generally move nasally (i.e., toward the nose) and/or superiorily (i.e., upward) as the pupil 106 constricts. Pupil constriction maybe referred to as miosis.

The example of FIG. 2 includes exemplary illustrations of the pupil 106 when dilated to first and second degrees of dilation 202 and 206, respectively. When dilated to the first degree of dilation 202, the pupil 106 is dilated to a greater extent than it is when dilated to the second degree of dilation 206.

The pupil centroid when the pupil 106 is dilated to the first degree of dilation 202 is illustrated by first X-shaped mark 210. The pupil centroid when the pupil 106 is dilated to the second degree of dilation 206 is illustrated by second X-shaped mark 214. Exemplary asterisk-shaped marks 218 may correspond to the first reference location, such as the center of the iris 110.

As can be seen from the example of FIG. 2, the pupil centroid 210 when the pupil 106 is dilated to the first degree of dilation 202 may be approximately 0.1 units in the X direction (i.e., 6.7−6.6), and the pupil centroid 214 when the pupil 106 is dilated to the second degree of dilation 206 may be approximately 0.4 units in the X direction (i.e., 6.7−6.3).

A location of the pupil centroid with respect to a second reference location may be referred to as pupil centroid shift. In other words, the pupil centroid shift may refer to a difference between the second reference location and the location of the pupil centroid. For example only, the second reference location may be the pupil centroid when the pupil 106 is fully dilated (e.g., taken before treatment) or another suitable reference location. The pupil centroid shift may be expressed as a multi-dimensional coordinate that relates the center of the pupil 118 to the second reference location. For example only, the pupil centroid shift may be expressed as a two-dimensional coordinate (e.g., X and Y) or a three-dimensional coordinate (e.g., X, Y, and Z) that relates the center of the pupil 118 to the second reference location. In the example of FIG. 2, assuming that the pupil centroid 210 corresponds to the second reference location, the pupil centroid shift may be approximately 0.3 units in the X direction (i.e., 0.4−0.1).

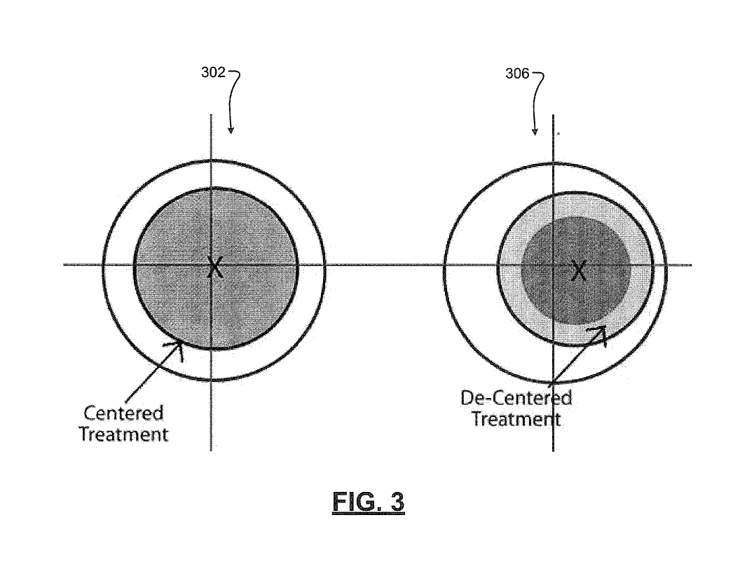

Failure to account for the pupil centroid shift associated with the varying pupil size during a vision correction treatment may result in the treatment being de-centered. A de-centered treatment may render a result of the vision correction treatment less than optimal inducing aberrations or imperfections. For example only, the pupil 106 may expand or contract during the treatment as the patient attempts to focus on a fixation target, as the emotional state of the patient changes (e.g., fear), as lighting conditions vary, and/or as one or more other conditions occur. An exemplary illustration of a centered treatment 302 versus a de-centered treatment 306 is presented in the example of FIG. 3.

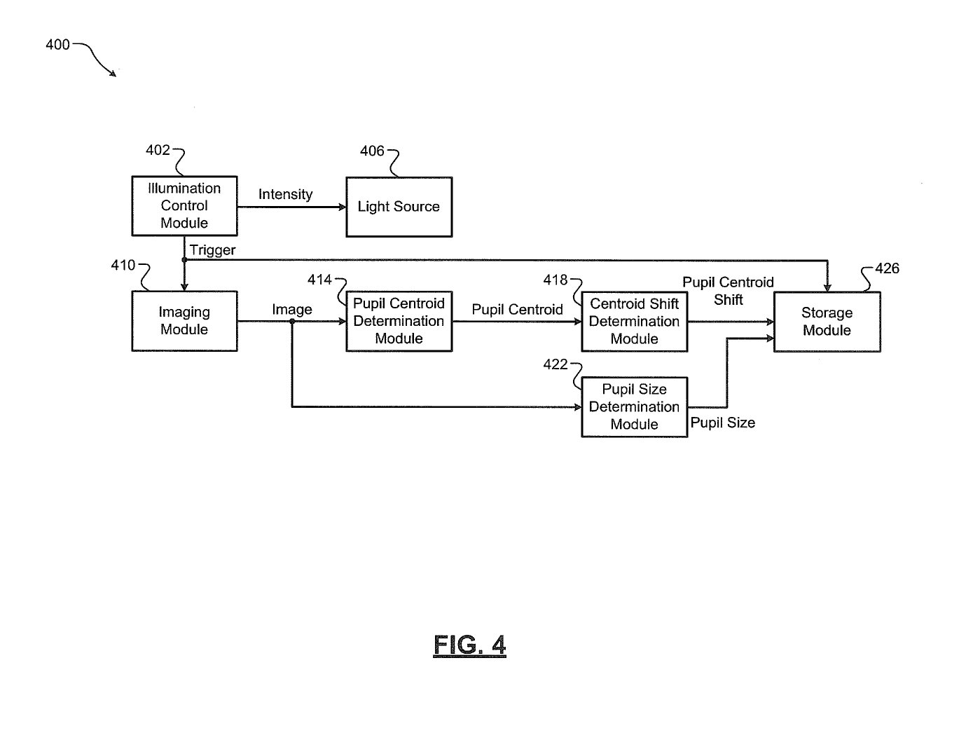

Referring now to FIG. 4, a functional block diagram of an exemplary pupil centroid shift determination and storage system 400 is presented. In various implementations, the pupil centroid shift determination and storage system 400 may be implemented with any device that can measure pupil size, such as a pupilometer, refractometer, or an aberrometer (e.g., a wavescan), with a treatment device that contains the necessary measuring hardware (e.g., a laser), with another suitable device, and/or independently.

An illumination control module 402 controls an intensity of light provided to the eye by a light source 406. For example only, the light source 406 may include one or more light emitting diodes (LEDs) and/or other suitable light sources. The illumination control module 402 may control the intensity of the light in a predetermined profile. For example only, the illumination control module 402 may vary the intensity of the light from a predetermined minimum intensity to a predetermined maximum intensity and back to the predetermined minimum intensity, vice versa, or in another suitable profile. The predetermined minimum intensity may correspond to a lighting condition that will cause the pupil 106 to be fully dilated. In other words, the predetermined minimum intensity may correspond to a lighting condition that will create a greatest pupil size. In contrast, the predetermined maximum intensity may correspond to a lighting condition that will cause the pupil 106 to constrict to a greatest extent. In other words, the predetermined maximum intensity may correspond to a lighting condition that will create a smallest pupil size. Varying the intensity of the light from the predetermined minimum intensity to the predetermined maximum intensity and back to the predetermined minimum intensity or vice versa may enhance a result of the treatment by predicting an associated pupil centroid shift to better maintain the orientation of the treatment laser relative to the patient's cornea. More specifically, as the patient's pupil size varies (i.e., increases or decreases), the proper frame of reference of the treatment laser can be maintained based on the pupil centroid and the associated pupil centroid shift.

The illumination control module 402 may vary the intensity in predetermined steps. For example only, the illumination control module 402 may increment or decrement the intensity of the light by a predetermined amount for each change in the intensity. The predetermined amount may correspond to a minimum lighting condition change that may create a measurable change (e.g., 0.01 mm) in pupil size.

The illumination control module 402 may trigger an imaging module 410 when the intensity of the light has been constant for at least a predetermined period. The predetermined period may correspond to a period of time after a change in the intensity at which the pupil size may be in a steady-state condition. The imaging module 410 may capture an image of the eye when triggered by the illumination control module 402. For example only, the image may be similar to the example of FIG. 1. The illumination control module 402 may increment or decrement the intensity of the light to a next intensity after the imaging module 410 captures the image. In this manner, the imaging module 410 may capture an image for each measurable pupil size.

A pupil centroid determination module 414 may determine the pupil centroid based on the image. The pupil centroid determination module 414 may determine the pupil centroid based on, for example, the location of the center of the pupil 106 with respect to the first reference location.

A centroid shift determination module 418 determines the pupil centroid shift based on the pupil centroid. For example only, the centroid shift determination module 418 may determine the pupil centroid shift based on the location of the pupil centroid with respect to the second reference location. A pupil size determination module 422 may determine the pupil size based on the image. For example only, the pupil size may include a radius of the pupil 106, a diameter of the pupil 106, or another suitable measurement of the size of the pupil 106.

When triggered by the illumination control module 402, a storage module 426 may store the pupil size and the pupil centroid shift. The storage module 426 may store the pupil centroid shift in a mapping (e.g., a look up table or LUT) by the pupil size. In other words, the storage module 426 may populate a mapping of pupil centroid shifts for the eye indexed by pupil size. In this manner, the mapping may include pupil centroid shifts for various pupil sizes, respectively.

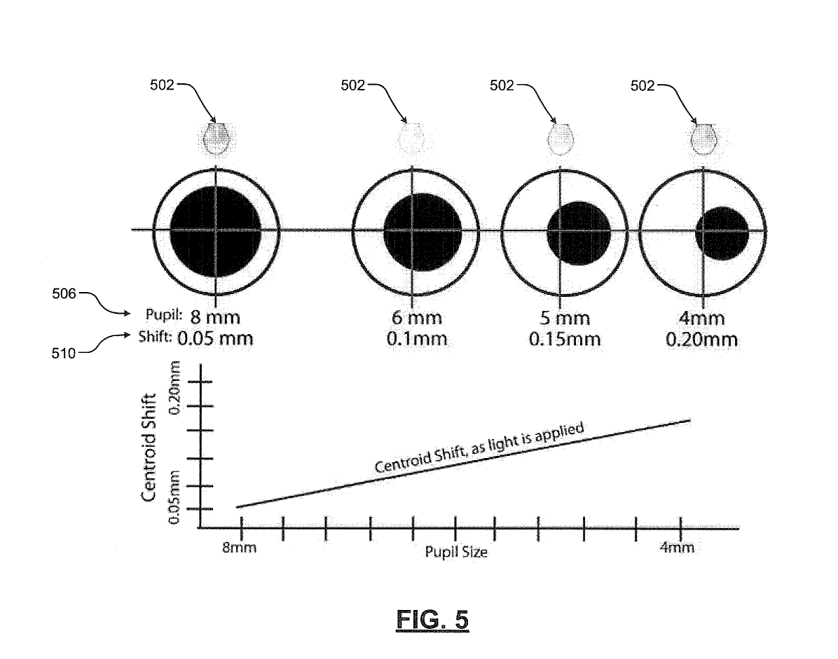

An exemplary graph of pupil centroid shift as a function of pupil size is presented in the example of FIG. 5. While the exemplary graph illustrates a linear relationship between pupil centroid shift and pupil size, the relationship may be non-linear and may take another suitable form. Additionally, while the exemplary graph illustrates a one dimensional relationship between pupil centroid shift (e.g., magnitude) and pupil size, the relationship between pupil centroid shift and pupil size may be multi-dimensional. The example of FIG. 5 also includes an exemplary illustration of exemplary light intensities 502 and associated exemplary pupil sizes 506 and exemplary pupil centroid shifts 510, respectively.

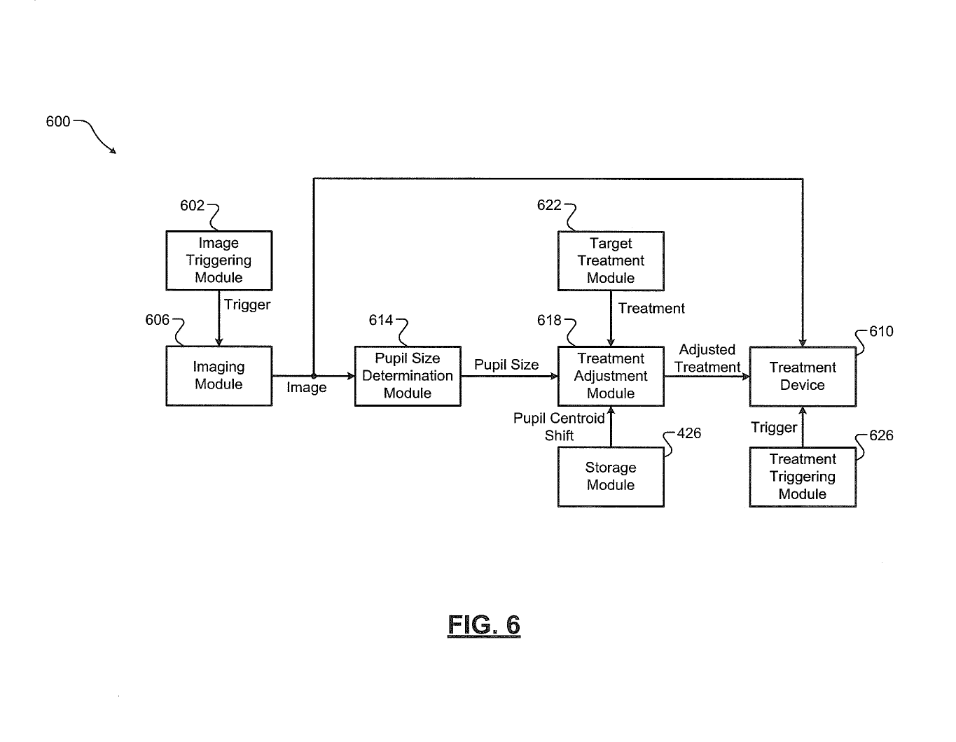

Referring now to FIG. 6, a functional block diagram of an exemplary vision treatment system 600 is presented. An image triggering module 602 may selectively trigger an imaging module 606 to take an image. The image triggering module 602 may trigger the imaging module 606 to take images at a predetermined frequency. The predetermined frequency may be set to greater than or equal to twice the treatment frequency of a treatment module 610 that performs the treatment. For example only, the treatment module 610 may include an excimer laser having a treatment frequency of approximately 20 Hz. In such an example, the predetermined frequency may be greater than or equal to 40 Hz. In various implementations, the predetermined frequency may be approximately 200 Hz.

When triggered, the imaging module 606 takes an image of the eye. For example only, the image may be similar to the example of FIG. 1. A pupil size determination module 614 determines the pupil size based on the image. A treatment adjustment module 618 receives the pupil size and a target treatment. In various implementations, the target treatment centroid shift compensation may be generated before the vision correction treatment is performed based on the data from an aberrometer.

Based on the determined pupil size, the treatment adjustment module 618 retrieves a pupil centroid shift associated with the pupil size. For example only, the treatment adjustment module 618 may retrieve the pupil centroid shift from the storage module 426 based on the pupil size. In various implementations, such as implementations where the pupil centroid shift determination and storage system 400 is implemented independently or with an aberrometer, the contents of the mapping may be made available (e.g., uploaded) to the vision treatment system 600 before the treatment.

The treatment adjustment module 618 may adjust the target treatment based on the pupil centroid shift and output an adjusted treatment for the eye. For example only, the target treatment may include a center for the target treatment. The treatment adjustment module 618 may adjust the target treatment by moving the center used during the treatment in a direction opposite to the pupil centroid shift. For example only, for a center with coordinates of 0 units in an X-direction and 0 units in a Y-direction (e.g., (0,0)) and for a pupil centroid shift with coordinates of 4 units in the positive X-direction and 3 units in the positive Y-direction (e.g., (4,3)), the treatment adjustment module 618 may adjust the target treatment by moving the center to 4 units in the negative X-direction and 3 units in the negative Y-direction (e.g., (−4, −3)).

A treatment triggering module 626 selectively triggers the treatment module 610. The treatment triggering module 626 may trigger the treatment module 610 at the treatment frequency. The treatment frequency may be a predetermined frequency, such approximately 20 Hz. When triggered, the treatment module 610 treats the eye based on the image and the adjusted treatment. In this manner, the pupil centroid shift is accounted for in real-time (actively) during the vision correction treatment.

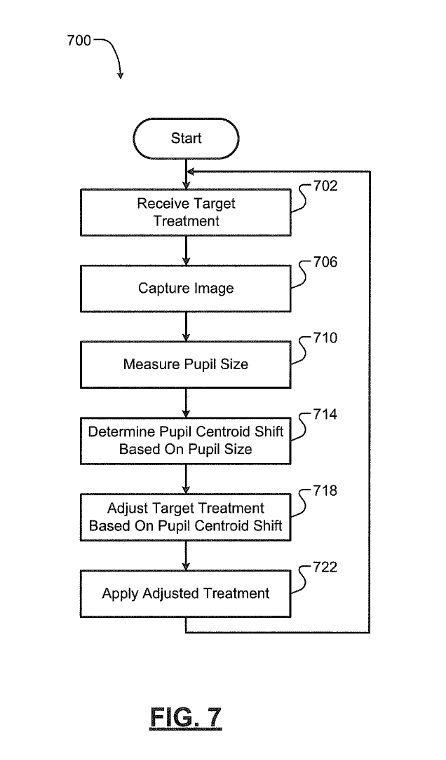

Referring now to FIG. 7, a flowchart depicting an exemplary method 700 of accounting for pupil centroid shift in real time (active) during a vision correction treatment of an eye is presented. Control may begin at 702 where control may receive a target treatment for the eye. Control may capture an image of the eye at 706.

At 710, control may measure a pupil size based on the image. Control may determine the pupil centroid shift based on the pupil size at 714. For example only, control may determine the pupil centroid shift from a mapping of pupil centroid shifts for the eye indexed by pupil size populated before the treatment. Control may adjust the target treatment based on the pupil centroid shift at 718. At 722, control may treat the eye based on the adjusted treatment, and control may return to 702. In this manner, control accounts for the possibility (and/or for the variability of the pupil size and the corresponding centroid shift) of variable pupil centroid shift in real time (active) during performance of a vision correction treatment.

The foregoing description of the embodiments has been provided for purposes of illustration and description. It is not intended to be exhaustive or to limit the invention. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the invention, and all such modifications are intended to be included within the scope of the invention.

What is claimed is:

1. A method for actively compensating for pupil centroid shift, said method comprising:
obtaining a first measurement of a first reference point relative to a predetermined reference frame, said first reference point being associated with a first pupil diameter of a patient;
obtaining a second measurement of a second reference point relative to said predetermined reference frame, said second reference point being associated with a second pupil diameter of the patient, said second pupil diameter being different from said first pupil diameter;
actively determining a relationship between said first measurement and said second measurement; and
actively generating a correction in response to said relationship, said correction being used by a treatment laser in association with an eye surgery.

2. The method according to claim 1 wherein said actively determining a relationship between said first measurement and said second measurement is continually completed during said eye surgery.

3. The method according to claim 1 wherein said actively generating a correction in response to said relationship, said correction being used by a treatment laser in association with an eye surgery comprising:
determining a third pupil diameter of the patient during the eye surgery, said third pupil diameter being different than said first pupil diameter and said second pupil diameter; and
generating said correction by comparing said third pupil diameter to said relationship.

4. The method according to claim 1 further comprising:
before said eye surgery, populating a mapping with values of said second measurement indexed by pupil diameter of the patient; and
obtaining said second measurement during said eye surgery based on said mapping and said second pupil diameter.

5. The method according to claim 1 further comprising:
capturing an image of an eye to be treated during said eye surgery; and
determining said second pupil diameter during said eye surgery based on said image.

6. A laser vision treatment system comprising:
an imaging module that captures an image of an eye of a patient during a laser vision correction procedure;
a pupil size determination module that determines a diameter of a pupil of the eye during the laser vision correction procedure;
a treatment adjustment module that receives a first location for centering the laser vision correction procedure, that determines a pupil centroid shift based on the diameter of the pupil during the laser vision correction procedure, and that determines a second location for centering the laser vision correction procedure based on the first location and the pupil centroid shift during the laser vision correction procedure; and
a treatment device that treats the eye and performs a portion of the laser vision correction procedure based on the second location.

7. The laser vision treatment system of claim 6 wherein the imaging module captures images of the eye during the laser vision correction procedure at a first predetermined frequency,
wherein the treatment device is capable of treating the eye during the laser vision correction procedure at up to a second predetermined frequency, and
wherein the first predetermined frequency is greater than the second predetermined frequency.

8. The laser vision treatment system of claim 7 wherein the first predetermined frequency is at least two times the first predetermined frequency.

9. The laser vision treatment system of claim 6 wherein the treatment device includes an excimer laser.

10. The laser vision treatment system of claim 6 wherein the treatment adjustment module determines the pupil centroid shift during the laser vision correction procedure using the diameter and a mapping of values of the pupil centroid shift indexed by values of the diameter measured before the laser vision correction procedure.

11. The laser vision treatment system of claim 10 further comprising:
a light source that emits first and second levels of light to the eye of the patient at first and second times before the laser vision correction procedure, respectively, wherein the first and second levels are different and the first and second times are different;
an imaging module that captures first and second images of the eye before the laser vision correction procedure while the light source is emitting the first and second levels of light, respectively;
a pupil size determination module that, before the laser vision correction procedure, determines first and second values of the diameter based on the first and second images, respectively and stores the first and second values in the mapping;
a pupil centroid determination module that determines first and second pupil centroids based on the first and second images before the laser vision correction procedure, respectively; and
a centroid shift determination module that, before the laser vision correction procedure, determines first and second values of the pupil centroid shift based on the first and second images, respectively, and indexes the first and second values of the pupil centroid shift in the mapping by the first and second values of the diameter, respectively.

12. The laser vision treatment system of claim 6 wherein the pupil centroid shift and the first location are expressed in two dimensions, the first location is expressed relative to a first frame of reference, and the pupil centroid shift is expressed relative to a second frame of reference centered at the first location, and
wherein the treatment adjustment module determines the second location by reflecting the pupil centroid shift in the two dimensions across the second frame of reference.
